(19) United States
(12) Patent Application Publication
(10) Pub. No.: US 2026/0077288 A1
(43) Pub. Date: Mar. 19, 2026

(54) AIR WASHER THAT FACILITATES THE REPLACEMENT OF THE FILTER ASSEMBLY

(71) Applicant: Quwei Tu, Shengzhou (CN)

(72) Inventor: Quwei Tu, Shengzhou (CN)

(21) Appl. No.: 18/890,673

(22) Filed: Sep. 19, 2024

(51) Int. Cl. B01D 46/00; A61L 9/20; B01D 46/10; B01D 46/42; B01D 53/02; F24F 8/22

(52) U.S. Cl. CPC B01D 46/0036; A61L 9/20; B01D 46/0028; B01D 46/0038; B01D 46/0045; B01D 46/10; B01D 46/4227; B01D 53/02; F24F 8/22; A61L 2209/12; A61L 2209/14; A61L 2209/15; B01D 2253/102; B01D 2257/406; B01D 2257/7027; B01D 2257/708; B01D 2257/90; B01D 2257/91; B01D 2258/06; B01D 2279/50; B01D 2279/65

(57) ABSTRACT

An air washer with easy replacement of filter components is disclosed, including a cover box and a bottom box. An air inlet is fixedly provided at middle of one side of an outer wall of the bottom box, and an air outlet cover is embedded in a centre of a top of the cover box. A filter is embedded at an upper end inside of the bottom box, a second filter plate is installed at an upper end of the filter, and a first filter plate is installed at an upper end of the second filter plate. An ultraviolet sterilization lamp is fixed on the upper end inside of the bottom box, and a centrifugal fan is fixedly provided at a middle of an inner bottom surface of the bottom box. The air washer can effectively improve the cleanliness of indoor and outdoor airs.

### TECHNICAL FIELD

The present application relates to the technical field of air washers, and in particular to an air washer with easy replacement of filter components.

### BACKGROUND

With improvement of people's requirements for indoor air quality, traditional air purifiers have been unable to meet some specific needs. These equipments often consist of motors, fans and filter devices that purify air by circulating the indoor air and filtering dust particle pollutants. However, the traditional air purifiers have limitations in dealing with spreading of aerosol bacteria.

In order to solve shortcomings of the traditional air purifiers in handling the spread of aerosol bacteria, engineers in existing technologies provide an air washer that facilitates the replacement of the filter components to solve the problems raised in the above.

### SUMMARY

To solve the shortcomings in the existing technologies, the present disclosure aims to provide an air washer with easy replacement of filter components that can effectively improve the cleanliness of indoor and outdoor airs and reduce the transmission opportunity of aerosol bacteria, thereby ensuring the respiratory health of users.

To achieve the above objectives, the present disclosure adopts the following technical solutions.

In some embodiments of the present disclosure, an air washer with easy replacement of filter components is provided, including a cover box and a bottom box. Herein an air inlet is fixedly provided at middle of one side of an outer wall of the bottom box, and an air outlet cover is embedded in a centre of a top of the cover box. A filter is embedded at an upper end inside of the bottom box, a second filter plate is installed at an upper end of the filter and a first filter plate is installed at an upper end of the second filter plate.

An ultraviolet sterilization lamp is fixed on one side of the upper end inside of the bottom box, and a centrifugal fan is fixedly provided at a middle of an inner bottom surface of the bottom box.

Further, the outer wall of the bottom box is fixedly provided with two support blocks on one side, and the two support blocks are located at front and rear of the outer wall of the bottom box.

Further, the cover box is embedded at an upper end of the bottom box, and the cover box is fixedly connected to the bottom box by a plurality of locks.

Further, the outer wall of the bottom box has a handle mounted on one outer wall opposite to one side where the support blocks being mounted and the handle is a convertible handle.

Further, the outer wall of the bottom box is mounted with a plug at a lower end where the handle being mounted and the plug is a triangular plug.

Further, the filter plate has a filter mesh structure for capturing larger particles.

Further, the second filter plate is an activated carbon filter plate for removing harmful substances and effectively absorbing noxious substances of formaldehyde, benzene and ammonia in indoor air.

Further, the centrifugal fan is driven by a brushless DC frequency conversion motor, and a filter mesh is embedded in an opening of the air inlet.

The air washer with easy replacement of filter components of the present disclosure has at least the following beneficial effects.

The air washer of the present disclosure which facilitates the replacement of the filter components, achieve an initial washing and secondary sterilization by the air sterilization filter set in the air outlet of the fan. This equipment can effectively improve the cleanliness of indoor and outdoor air, thereby reducing the transmission opportunity of aerosol bacteria, and also ensuring the respiratory health of users. At the same time, the brushless DC frequency conversion motor is combined with a curve centrifugal fan with the volute contour, as the centrifugal fan adopts the volute line structure, which makes the air inlet and exhaust air more powerful. The cover box uses a number of locks to quickly complete the splicing and installation of the bottom box, so that facilitate the rapid disassembly and installation, and then realize the replacement and cleaning of the internal filter components.

### BRIEF DESCRIPTION OF THE DRAWINGS

FIG. 1 shows a schematic diagram of an axis-side structure of an air washer with easy replacement of filter components in accordance with some embodiment of the present disclosure.

FIG. 2 shows a schematic diagram of another axis side structure of the air washer with easy replacement of filter components in accordance with some embodiment of the present disclosure.

FIG. 3 shows a schematic diagram of a local explosion structure of the air washer with easy replacement of filter components in accordance with some embodiment of the present disclosure.

FIG. 4 shows another schematic diagram of the local explosion structure of the air washer with easy replacement of filter components in accordance with some embodiment of the present disclosure.

FIG. 5 shows a schematic diagram of an explosion structure from another view of the air washer with easy replacement of filter components in accordance with some embodiment of the present disclosure.

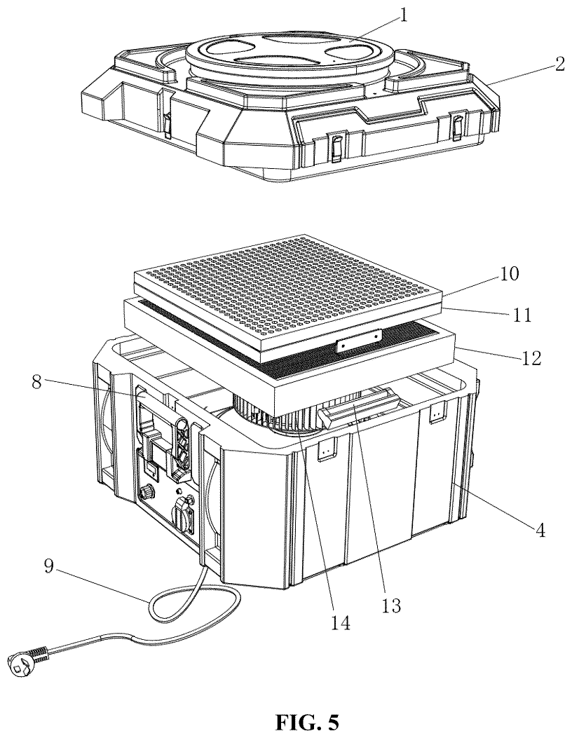

In the drawings, reference signs are as follows. 1. Air outlet cover, 2. Cover box, 3. Lock, 4. Bottom box, 5. Air inlet, 6. Filter, 7. Support block, 8. Handle, 9. Plug, 10. First filter plate, 11. Second filter plate, 12. Filter, 13. Ultraviolet sterilization lamp, 14. Centrifugal fan.

### DETAILED DESCRIPTION OF THE EMBODIMENTS

The technical solutions in the embodiments of the present disclosure will be clearly and completely described in following in combination with the accompanying drawings in the embodiments. Obviously, the described embodiments are only a part of the embodiments rather than all embodiments. Based on the embodiments in the present disclosure, all other embodiments obtained by one ordinary skill in the art without making creative labor shall fall within the scope of the protection of the present invention.

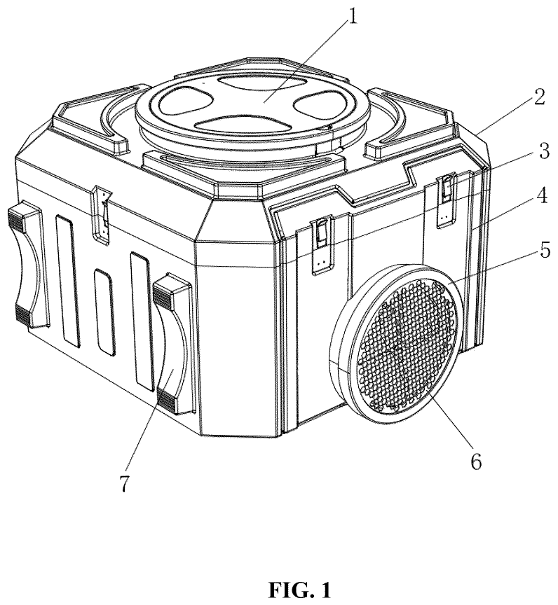

Referring to FIG. 1 to FIG. 4, in some embodiments of the present disclosure, an air washer with easy replacement of filter components is provided, including a cover box 2 and a bottom box 4. An air inlet 5 is fixedly provided at the middle of one side of an outer wall of the bottom box 4, and an air cover 1 is embedded in a centre of the top of the cover box 2. A filter 12 is embedded at an upper end inside of the bottom box 4, a second filter plate 11 is installed at the upper end of the filter 12, and a first filter plate 10 is installed at an upper end of the second filter plate 11.

An ultraviolet sterilization lamp 13 is fixed at the upper end inside of the bottom box 4, and a centrifugal fan 14 is fixed at the centre of an inner bottom surface of the bottom box 4.

Specifically, the internal ultraviolet sterilization lamp 13 can be used by the ultraviolet lamp to eliminate bacteria and viruses in the air, and the filter plate and filter 12 can effectively improve the cleanliness of the indoor and outdoor air, reduce the transmission opportunity of aerosol bacteria, and ensure the respiratory health of the user, and the upper air cover 1 has a plurality of air outlets to realize the discharge effect of the internal filtered air.

The first filter plate 10 has a filter mesh structure for capturing large particles, such as dust and hair. The second filter plate 11 is an activated carbon filter plate for removing harmful substances an, effectively adsorbing noxious substances of formaldehyde, benzene and ammonia in indoor air, thereby reducing the harm of these pollutants to human body. In addition, it also improves the indoor air quality by removing odors, such as smoke, musty, odor, while the internally set filter 12 usually uses HEPA filters that capture smaller particles such as bacteria and viruses.

Referring to FIG. 1, two support blocks 7 are fixed on one side of the outer wall of the bottom box 4, and the two support blocks 7 are located at front and rear of the outer wall of the bottom box 4. The cover box 2 is embedded in the upper end of the bottom box 4, and the cover box 2 is fixedly connected to the bottom box 4 by a plurality of locks 3.

Specifically, the support blocks 7 of the outer wall can be mounted, with the effect of installing the base, and the cover box 2 of the upper end is quickly assembled with multiple locks 3, so as to facilitate the rapid disassembly and installation, and then realize the replacement and cleaning of the internal filter components.

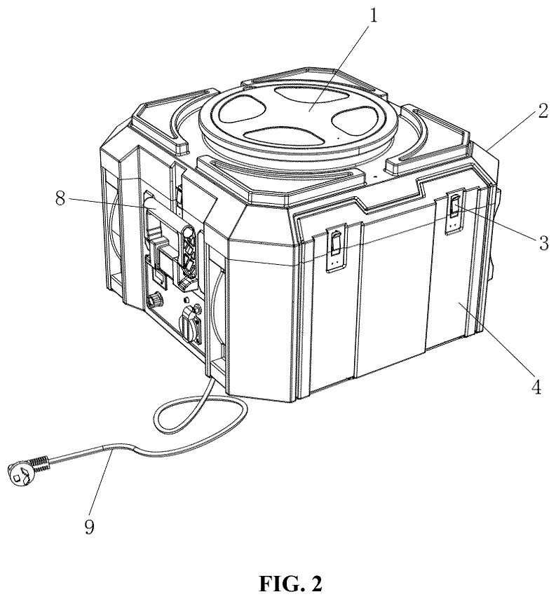

Referring to FIG. 2, the outer wall of the bottom box 4 is mounted with a handle 8 on the side opposite the support block 7, and the handle 8 is a inverted handle 8; the outer wall of the bottom box 4 is mounted at the lower end of the handle 8, and the plug 9 is a triangular plug.

Specifically, when not in use, the handle 8 can be turned to the upper end in contact with the outer wall of the bottom box 4 to reduce space occupation. In the need of handling, the handle 8 can be turned down to make it a 90° structure, at this time, the handle 8 can complete the lifting or handling and use. Use the hand end limit a plug 9 to connect to the socket to realize the power supply.

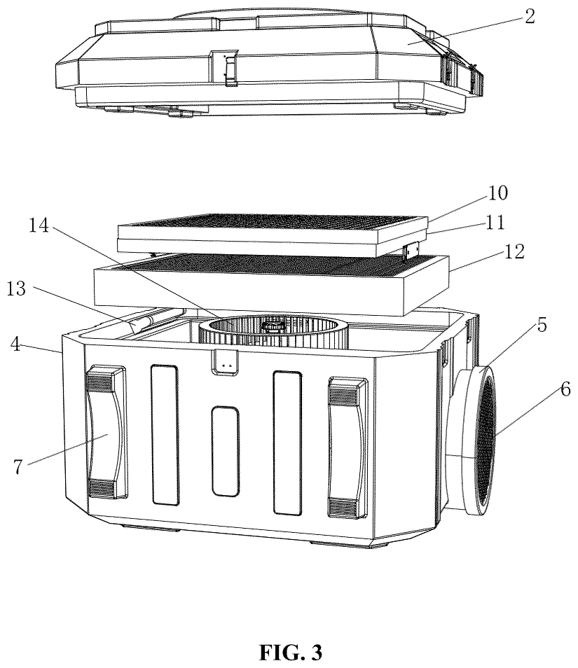

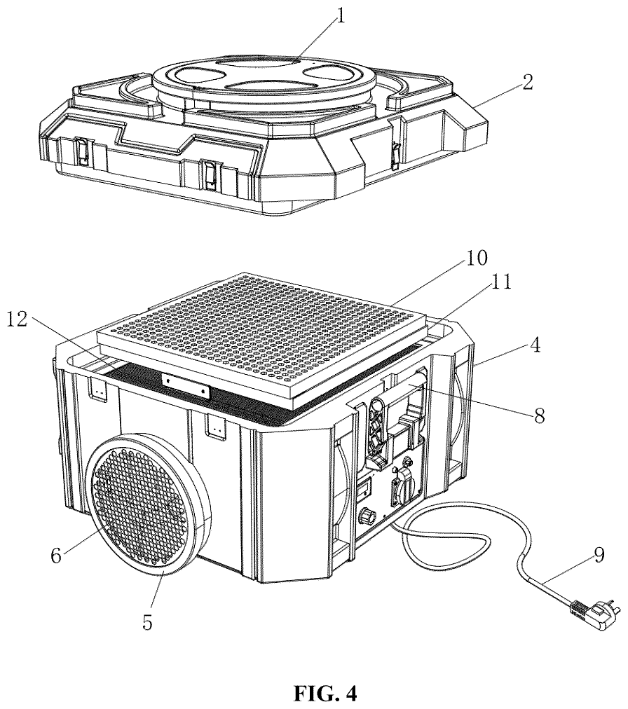

With reference to FIG. 1, FIG. 3 and FIG. 4, the centrifugal fan 14 is driven by using a brushless DC frequency conversion motor, and the filter 6 is embedded at the opening of the air inlet 5. The filter 6 disposed inside the inlet 5 may avoid the blockage caused by external debris entering the inlet 5 and reduce the probability of damage.

And brushless DC motor does not use mechanical brush device, using square wave automatic control type permanent magnet synchronous motor, replace the carbon brush with hall sensor, NdFeB as a rotor permanent magnet material, compared with the general traditional dc motor it has high efficiency, good stability, low noise and long life, with volute contour curve air duct system of centrifugal fan 14 make the air pressure volume greatly improved beyond the traditional motor air washer performance.

Working principle: in use, the centrifugal fan 14 work is the gas suction machine, through the centrifugal fan 14 high speed rotation produce centrifugal force, and the gas out of the fan, the realization of gas pressure increase and flow change process, compared with the traditional axial fan wind volume is smaller, and centrifugal fan 14 air volume wind pressure is larger and centrifugal fan 14 exhaust distance and air distance are far.

Secondly, the gas is discharged from the upper outlet cover 1, and the outlet port is provided on the outlet cover 1, and the filter 12, the filter plate 10, the filter plate 10 and the filter plate 11 can realize the primary washing and secondary sterilization of the air, thus effectively improving the cleanliness of the indoor and outdoor air.

Finally, it should be noted that the above is only the preferred embodiment of the utility model, and is not used to limit the utility model. Despite the detailed description of the technical scheme described in the aforementioned embodiment of the utility model, any modification, equivalent replacement or improvement of the utility model shall be included within the protection scope of the utility model.

What is claimed is:

1. An air washer with easy replacement of filter components, comprising a cover box and a bottom box;
wherein an air inlet is fixedly provided at middle of one side of an outer wall of the bottom box, and an air outlet cover is embedded in a centre of a top of the cover box;
wherein a filter is embedded at an upper end inside of the bottom box, a second filter plate is installed at an upper end of the filter, and a first filter plate is installed at an upper end of the second filter plate; and
wherein the bottom box is fixed with an ultraviolet sterilization lamp on one side of the upper end inside, and a centrifugal fan is fixedly provided at a middle of an inner bottom surface of the bottom box.

2. The air washer with easy replacement of filter components according to claim 1, wherein two support blocks are fixedly provided on the one side of the outer wall of the bottom box, and the two support blocks are located at front and rear of the outer wall of the bottom box.

3. The air washer with easy replacement of filter components according to claim 1, wherein the cover box is embedded at an upper end of the bottom box, and the cover box is fixedly connected to the bottom box by a plurality of locks.

4. The air washer with easy replacement of filter components according to claim 1, wherein the outer wall of the bottom box has a handle mounted on one outer wall opposite to one side where the support blocks being mounted, and the handle is a convertible handle.

5. The air washer with easy replacement of filter components according to claim 1, wherein the outer wall of the bottom box is mounted with a plug at a lower end where the handle being mounted and the plug is a triangular plug.

6. The air washer with easy replacement of filter components according to claim 1, wherein the filter plate has a filter mesh structure for capturing larger particles; and
wherein the second filter plate is an activated carbon filter plate for removing harmful substances and effectively absorbing noxious substances of formaldehyde, benzene and ammonia in indoor air.

7. The air washer with easy replacement of filter components according to claim 1, wherein the centrifugal fan is driven by a brushless DC frequency conversion motor and a filter mesh is embedded in an opening of the air inlet.
